(19) United States
(12) Patent Application Publication
(10) Pub. No.: US 2018/0325721 A1
(43) Pub. Date: Nov. 15, 2018

(54) DENTAL ALIGNER, METHOD OF PRODUCTION, AND ORTHOSIS COMPRISING SUCH AN ALIGNER

(71) Applicant: OSCIMED SA, La Chaux-de-Fonds (CH)

(72) Inventor: Jacques MAGNIN, La Chaux-de-Fonds (CH)

(73) Assignee: OSCIMED SA, La Chaux-de-Fonds (CH)

(21) Appl. No.: 15/775,555

(22) PCT Filed: Nov. 4, 2016
(86) PCT No.: PCT/EP2016/076724

§ 371 Date: May 11, 2018

(30) Foreign Application Priority Data
Nov. 16, 2015 (EP) 15194779.3

(51) Int. Cl. A61F 5/56

(52) U.S. Cl. CPC A61F 5/566; A61F 2005/563

(57) ABSTRACT

A dental aligner includes a framework which is made of a rigid synthetic material and supports a shell made of thermoformable material. The framework includes an anterior wall, which is referred to as the labial wall, and a posterior wall, which is referred to as the lingual wall. The walls are substantially parallel to each other and are connected by a bottom wall, which is referred to as the occlusal wall, the latter being substantially perpendicular to both the labial wall and the lingual wall in such a way that the framework has a U-shaped cross section in any vertical plane. The thermoformable shell is accommodated between the labial, lingual and occlusal walls of the framework essentially and is anchored thereon by way of anchoring means which are formed in the framework and the thermoformable shell.

### TECHNICAL FIELD

The present invention relates to the field of oral care equipment for the treatment and/or prophylaxis of dental, mandibular and/or respiratory pathologies and conditions in humans. It relates more particularly to a dental aligner for adjusting and optimising the bite and creating necessary space in the mouths of patients.

### STATE OF THE ART

Dental aligners (occlusal and mandibular) as well as devices comprising such aligners belong to the group of oral orthotics which also includes mouthguards, the apparatus and equipment for treating sleep apnea, and intra—oral orthopedic devices that are appropriate for the treatment of dysfunctions of the masticatory system (DMS), such as bruxism for example, but also other respiratory indications such snoring and sleep apnea.

Bruxism is a common condition more generally known as “teeth grinding” which consists of the undesirable gnashing/grinding and/or clenching of teeth outside of periods of regular chewing or swallowing, during which the upper- lower teeth are naturally in contact. These phases of teeth grinding or clenching may occur diurnally and/or nocturnally. Whereas the diurnal phases generally do not cause problems and are related to natural jaw relaxing mechanisms, the nocturnal phases, which are uncontrolled, can lead to various consequences that are more or less harmful or detrimental to the individual subjects of this pathology ; these include :
wearing of the enamel and, where applicable of the dentin (underlying layer of ivory yellowish tissue);
increased sensitivity of the teeth to variations in temperature and acidity of ingested foods and liquids;
tooth fractures;
postural perturbations.

To date being fitted with an occlusal dental aligner constitutes the preferred treatment for bruxism, designed primarily to prevent and limit tooth wear, in particular.

Snoring, also referred to as ronchopathy, is a pathology of the respiratory system that is linked to muscle and tissue relaxation at the mandible and inside of the mouth during sleep. This relaxation brings about a withdrawal of the tongue to the entrance of the trachea that causes disturbances of respiratory flow and the phenomena of vibration of the uvula and of the soft tissue of the palate responsible for causing a noise, which is characteristic of snoring.

Snoring in a majority of cases, constitutes a benign perturbation or disturbance of respiration and sleep in individuals who are the affected subjects, or even their near and dear when sound levels, which can reach 100 dB, are high. However, in some individuals, it may also be the source of more significant disturbances in particular respiratory interruptions that disrupt sleep. In such an event, it is referred to as sleep apnea. Apnea usually causes fatigue and daytime drowsiness, as well as increased risks of hypertension and cardiovascular diseases such as, in particular, cerebrovascular disorders or conditions and infarction.

In order to counter snoring and sleep apnea, intra-oral devices have been proposed, that comprise at least one mandibular aligner configured in order to promote a frontward directed movement (posterior-anterior) of the mandibule during sleep, in a manner so as to compensate the muscle relaxation and limit the retreating of the tongue towards the pharynx and the trachea, the latter being the main cause of respiratory obstruction which is the source of snoring and sleep apnea.

Various different configurations of occlusal and/or mandibular aligners as well as orthoses comprising such aligners have been proposed in the prior art, such as for example in the patent applications EP 1 602 347 A1 and EP 1 750 629 A2.

These applications in particular disclose mandibular advancement orthoses comprising two aligners that are adapted to the upper dental arch and the lower dental arch of the jaw and connected to each other in a manner so as to promote movement of the lower aligner and of the mandible by supporting the aligner on the corresponding dental arch in the posterior-anterior direction. The aligners of these orthoses comprise on the one hand, a thermoformable shell in the particular configuration of the dental arches and dental implants of the user of the orthosis as well as a framework which is made of a substantially rigid material supporting or over-moulded in the thermoformable shell. The rigid framework of the aligners presents a generally U-shaped form that is consistent with a dental arch and comprises anterior and posterior walls connected at their base by a bottom wall thus conferring to the framework a vertical cross section that is also U-shaped in a manner such that the said framework (and the thermoformable shell) is able to cover and fit over each of the dental arches by covering the posterior and anterior faces of the latter as well as the occlusal plane of each one thereof. The rigid framework of the aligners is also articulated by means of slots formed symmetrically in its anterior and posterior walls as well as at least partially in its bottom wall. These slots allow for a deformation of the rigid framework simultaneously with the thermoformable shell of each of the aligners so as to adjust and fit to the dental conformation. The result thereof are aligners and orthoses that it is possible to produce in large quantities while being individually conformable to the particular configuration of the dental arches of each patient, in order to provide for increased comfort and effectiveness of the orthosis and its aligners.

However, the known aligners disclosed in the prior art, in spite of being adaptable and adjustable to the particular conformation of each of the dental arches of a patient by virtue of the thermoformable nature of their shell and the articulated nature of their framework, are not entirely satisfactory in terms of comfort and ease of use for patients. Indeed, these aligners represent a significant dimensional impact with respect to the dental arches, in particular because of the complete over-moulding of the framework within the thermoformable shell, which causes discomfort for users both on the anterior labial faces of the dental arches, between the teeth and the lips, as well ason the posterior lingual faces of the dental arches between the teeth and the palate or the tongue.

The US patent application US 2013/081638 in addition also describes a dental aligner equipped with a rigid framework and a thermoformable shell positioned thereon in which the framework comprises on an internal face of the framework, a number of blind cavities intended for enhancing the stabilisation by calibration of the thermoformable shell in the framework. However, this stabilisation is in practice not sufficient to procure an effective stabilisation over time, with the thermoformable shell getting loosened/dislodged gradually from the floor of the framework as a result of the action and efforts of the jaw, particularly during teeth-grinding by the patient.

One objective of the present invention is therefore to provide a dental aligner that is free of the disadvantages of the prior art.

Another objective of the present invention is to provide a kit and a form setting method for forming such a dental aligner that provides the ability to all patients to themselves prepare an aligner according to the invention that is adapted to the individual conformation of one of their dental arches.

Another objective of the invention finally also consists in providing an orthosis that incorporates an aligner in accordance with this invention.

### DISCLOSURE OF THE INVENTION

These objectives are achieved according to one object of the invention by a dental aligner comprising a framework which is made of a rigid synthetic material and supports a shell made of thermoformable material, the said framework having an anterior wall, which is referred to as the labial wall, and a posterior wall, which is referred to as the lingual wall, which walls are substantially parallel to each other and are connected by a bottom wall, which is referred to as the occlusal wall, the latter being substantially perpendicular to the two labial and lingual walls in such a way that the framework has a U-shaped cross section in any vertical plane, the said thermoformable shell is accommodated between the said labial, lingual and occlusal walls of the said framework and characterised in that the thermoformable shell is anchored thereon by way of anchoring means which are formed in the framework and the thermoformable shell and having holes going therethrough from side to side between an internal surface of the occlusal wall on which rests the thermoformable shell and an external surface of the occlusal wall.

The dental aligner according to the invention is thus arranged in a manner such that the thermoformable shell thereof is essentially accommodated between the walls of the framework of the aligner and not predominantly by over-moulding thereof as in the prior art. Indeed, a complete over-moulding of the framework by the thermoformable shell is not in itself necessary to the functionality of the aligner but greatly increases its dimensional impact in the mouths of patients.

In addition, the aligner of the invention advantageously compensates for the risk of loosening/dislodging of the thermoformable shell from the framework by the presence of anchoring means for anchoring the shell on the framework, anchoring means effectuating a localised connection of the thermoformable shell on the framework, at positions selected in order to minimally disrupt the positioning of the aligner in the mouth and to reduce to the maximum extent the dimensional impact thereof.

According to one embodiment of the aligner of the invention, the anchoring means comprise at least one connecting tab covering at least partially, at least one segment of the labial wall and/or the lingual wall, the said connecting tab being formed integrally together with the thermoformable shell in a manner so as to crimp the said at least one segment of the labial wall and/or the lingual wall between the thermoformable shell and the connecting tab.

According to one embodiment, the holes formed through and through between an internal surface of the occlusal wall on which rests the thermoformable shell, have at least one dimension, taken in at least one vertical cross section of the occlusal wall, which is smaller at the level of the internal surface as compared to the corresponding dimension at the level of the external surface.

This particular configuration of the holes ensures the formation of pins made from the thermoformable material of the thermoformable shell through the holes, it not being possible for the said pins to be withdrawn into the shell once they have been inserted into the holes on account of the expansion of the thermoformable material of the shell towards the external surface of the framework, into the largest dimension of the holes.

Preferably, the said holes have a dimension that is variable on an increasing basis over the thickness of the occlusal wall between the internal surface and the external surface of the said wall.

Preferably, according to this particular embodiment, the said holes have a cross section, taken in the said vertical cross section of the occlusal wall, that is at least partly triangular or trapezoidal.

In one embodiment of the invention, the said framework is flexible on both sides of at least one perpendicular vertical plane of its labial, lingual and occlusal walls.

As may be applicable, in one embodiment, the said framework is flexible on both sides of a plurality of vertical planes that are perpendicular to the occlusal wall thereof.

In one embodiment of the invention, the said framework comprises at least one favoured zone of flexion formed by localised reduction of the constituent material of the framework.

Preferably, the said at least one favoured zone of flexion extends in at least one said vertical plane on either side of which the said framework is flexible.

Even more preferably, the at least one favoured zone of flexion comprises a recess, in particular a slot, formed in at least one labial or lingual wall and the occlusal wall of the framework.

According to one advantageous embodiment, the at least one favoured zone of flexion comprises a recess formed exclusively in the lingual wall and the occlusal wall.

According to another advantageous embodiment, the aligner of the invention comprises a plurality of flexion zones constituted by integrally formed recesses or indents located in the lingual and occlusal walls.

According to one variant of the embodiment, the object of the invention also relates to a dental aligner, comprising a framework which is made of a rigid synthetic material and supports a shell made of thermoformable material, the said framework having an anterior wall, which is referred to as the labial wall, and a posterior wall, which is referred to as the lingual wall, which walls are substantially parallel to each other and are connected by a bottom wall, which is referred to as the occlusal wall, the latter being substantially perpendicular to the two labial and lingual walls in such a way that the framework has a U-shaped cross section in any vertical plane, the said thermoformable shell is accommodated between the said labial, lingual and occlusal walls of the said framework, and characterised in that the thermoformable shell is anchored on to the framework by way of anchoring means that comprise at least one connecting tab extending at least partially into a recess, in particular a slot, formed in at least one labial or lingual wall and the occlusal wall of the framework, the said connecting tab being formed integrally together with the thermoformable shell so as to crimp the framework between the thermoformable shell and the connecting tab, the said recess constituting a zone of flexion of the aligner, the said framework on both sides of at least one vertical plane being perpendicular to its occlusal wall.

Thus in this embodiment of the invention, the anchoring means, integrated into the framework of the aligner in the form of a recess, for instance a slot at least procures both a function of anchoring and of flexibilising of the aligner in order to improve the comfort and the grip thereof in the mouth of a patient.

According to one embodiment, the said framework is flexible on both sides of a plurality of vertical planes that are perpendicular to the occlusal wall thereof.

In addition, the said framework preferably comprises a plurality of recesses each extending in at least one said vertical plane on either side of which the said framework is flexible.

Even more preferably in this embodiment the at least one recess is formed exclusively in the lingual wall and the occlusal wall of the framework.

According to a second object, the present invention relates to an adjustment method for adjusting a dental aligner as previously described above, comprising the following steps:
Heating of the dental aligner in a water bath at a temperature comprised between 60° C. and 90° C. in order to soften the shell which is made of thermoformable material for a period of at least 1 min;
Positioning of the dental aligner on an occlusal plate that is conformed substantially in a Y shape, the occlusal plate having one manual gripping arm and two support arms for supporting the dental aligner, which are attached at one of the ends thereof and at one end of the manual gripping arm, as well as wedging pads for wedging the said dental aligner, which are fittingly adjusted on at least one face of the said occlusal plate;
Insertion of the occlusal plate carrying the dental aligner in the mouth of a patient;
Form setting the thermoformable shell through application of pressure by one dental arch of the patient into the softened thermoformable shell and counter pressure on the occlusal plate by the opposite dental arch in order to procure an effect of formwork and of penetration of the softened material of the shell into the holes and/or recesses of the anchoring means of the framework for forming the said anchoring means;
Removing of the occlusal plate and the dental aligner from the mouth of the patient.

The method of the invention makes it possible by way of the effective implementation of an occlusal plate supporting the aligner of the present invention to provide for the taking of a dental impression and the form setting of the aligner in an extremely simple process, while also controlling the leaking of thermoformable material through the various holes or slots formed in the framework of the aligner of the invention. Indeed, during the impression taking, the occlusal plate provides a surface for formwork or for pushing back the material of the thermoformable shell in order to force the same in between the walls of the framework without overflowing on to the exterior of the walls of the framework. However, the effect of formwork makes it possible to simultaneously create an effective anchoring at the holes and/or recess(s) formed in the framework in the guise of anchoring means while ensuring the constraining of the thermoformable material crimped on the rims of the said holes and/or recess(s).

Where appropriate, the method of the invention may further comprise an additional step of cooling of the dental aligner after removal of the occlusal plate from the mouth of the patient.

The method of the invention may also comprise an additional step of separation of the thermoformable shell from the flexible framework in order to provide the patient with a simple anti-teeth grinding aligner, that is devoid of the framework. This separation step, however, induces the at least partial breaking of the anchoring means, in an irreversible manner.

According to a third object, the present invention also relates to a kit for operationally implementing a method as defined here above, that comprises an occlusal plate having one manual gripping arm and two support arms for supporting the dental aligner, which are attached to one of the ends thereof and to one end of the manual gripping arm as well as wedging pads for wedging the said aligner, which are adjusted on at least one face of the said occlusal plate and one dental aligner according to the invention. The combination of an aligner in accordance with the invention and an occlusal plate that is adapted so as to support the said aligner during the forming thereof provides the means for easy implementation for patient of the form setting method as previously described here above, without having to make use of a third party equipment.

According to a fourth object, the invention also provides a mandibular orthosis comprising at least one dental aligner as previously defined here above.

### BRIEF DESCRIPTION OF DRAWINGS

Further details of the invention will become more clearly apparent from reading the description that follows with reference being made to the accompanying drawings in which:

FIG. 1 represents an overall view of a dental aligner according to invention;

FIG. 2 represents a right side view of the aligner shown in FIG. 1;

FIG. 3 represents a view from the anterior face (labial) of the aligner shown in FIG. 1;

FIG. 4 represents a view from the posterior face (lingual) of the aligner shown in FIG. 1;

FIG. 5 represents a top view of the aligner shown in FIG. 1;

FIG. 6 represents a vertical cross section AA according to a sagittal plane M of the aligner shown in FIG. 3 or FIG. 4;

FIG. 7 represents a vertical cross section BB along a vertical plane B shown in FIG. 5;

FIG. 8 represents an overall view of a kit for making/producing an aligner as shown in FIG. 1;

FIGS. 9 to 12 represent various views of a mandibular orthosis comprising at least one dental aligner according to the invention.

### EMBODIMENT OF THE INVENTION

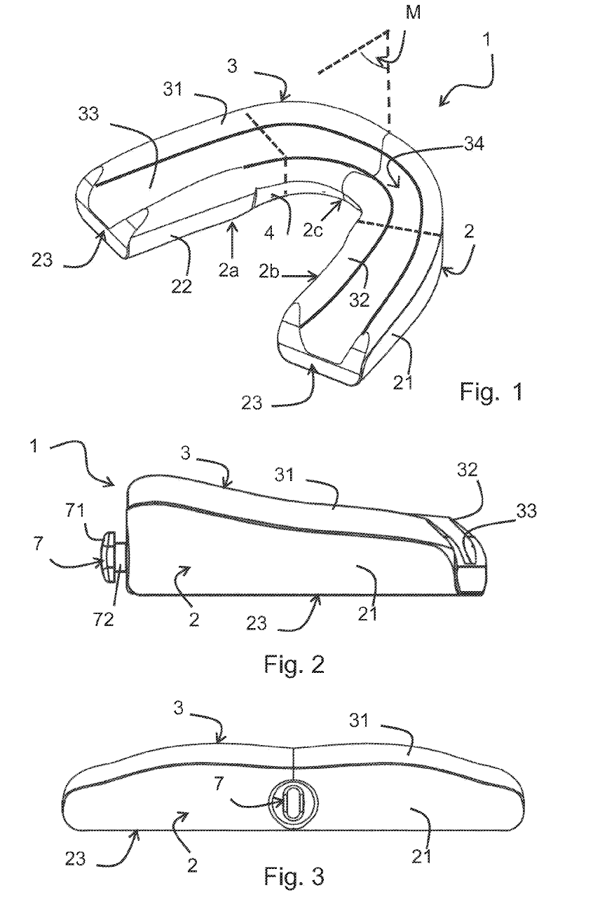

FIGS. 1 to 7 illustrate a dental aligner 1 according to one embodiment of the invention in different views thereof.

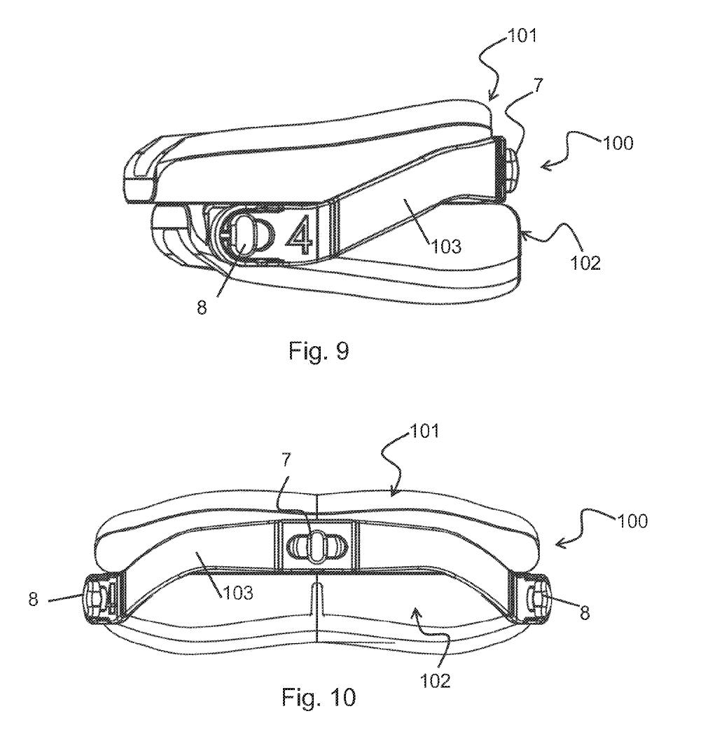

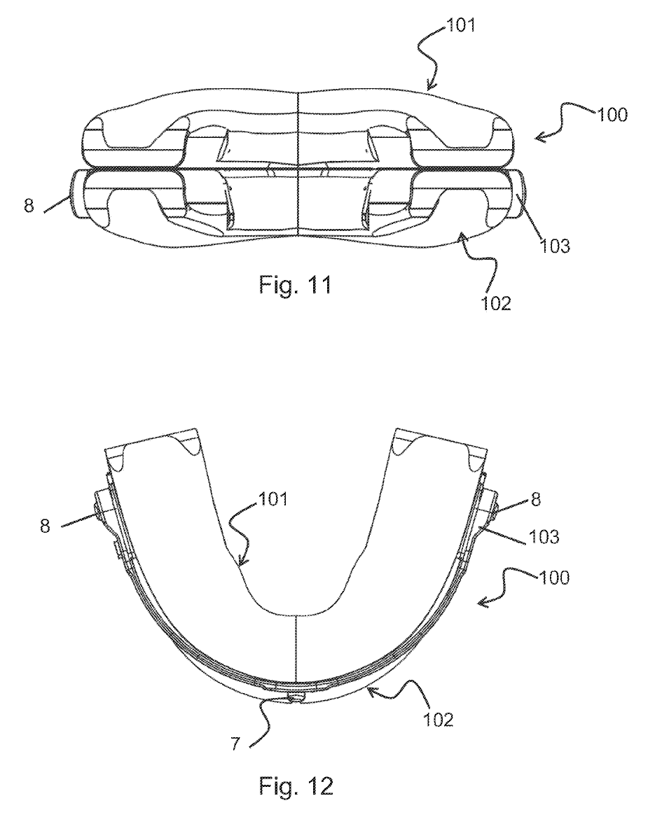

Such a dental aligner 1 may advantageously be used as an occlusal aligner and/or as a mandibular aligner, as may be appropriate in the context of a mandibular orthosis as represented in FIGS. 9 to 12.

As represented in FIGS. 1 to 7, the dental aligner 1 of the invention in a conventional manner is composed of a framework 2 and at least one thermoformable shell 3 that is supported and held in place on the framework 2.

The framework 2 is preferably constituted of a synthetic material that is biocompatible and/or at least of substantially rigid food grade quality. In particular, the framework 2 may be comprised of polyether block amide or polyester for example and formed by moulding or injection.

In a conventional manner the framework 2 has a shape and form analogous to that of a dental arch, that is to say, a substantially U-shaped form and includes two straight lateral sections 2a, 2b that are connected to each other by a curved central section 2c. The straight sections 2a, 2b are so conformed in order to, during normal use of the dental aligner 1, cover the posterior lateral fields of a dental arch of a human patient comprising of the premolars and molars when the central curved section is intended to cover the frontal field of the dental arch and the incisors and canines of the patient, quite obviously considering a full and regular set of teeth.

The framework 2 in addition has, here as well in a conventional manner, a substantially vertical anterior wall, which is referred to as the labial wall 21 and a vertical posterior wall, which is referred to as the lingual wall 22. The labial wall 21 and lingual wall 22 are connected to each other by a third wall of the framework, that is to say a bottom wall, which is referred to as the occlusal wall 23, that extends perpendicularly to the labial wall 21 and lingual wall 22 and connects them at their base.

It is understood here that the labial wall 21 is a wall of the framework 2 of the dental aligner 1 which, in normal use of the said dental aligner 1 is located between the dental arch of the patient and a lip of the latter. In a similar fashion, the term lingual wall 22, is understood to refer to a wall of the framework 2 of the dental aligner 1 which, in normal use of the said dental aligner 1 is located between the dental arch of the patient and the tongue or the palate of the latter. Finally, the term occlusal wall 23, is understood to refer to a wall of the framework 2 of the dental aligner 1 which, in normal use of the said dental aligner 1 is located in and/or defines an occlusal plane between the teeth of the patient, or at least one wall delimiting a contact interface for contact between the teeth of the dental arches of the patient.

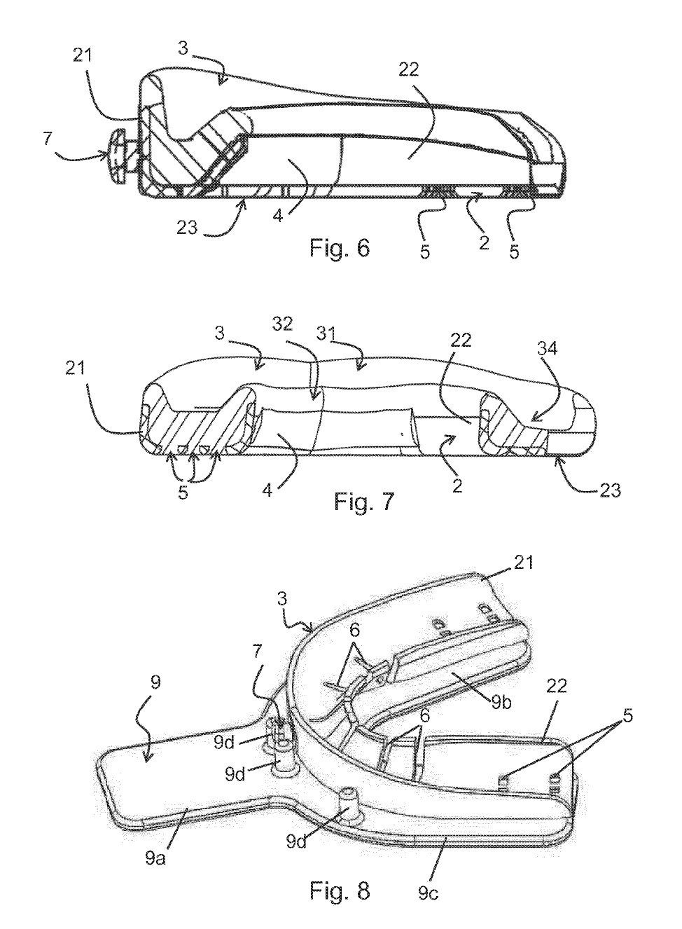

Thus, the labial wall 21 and lingual wall 22 are substantially parallel to each other and substantially perpendicular to the occlusal wall 23 in a manner such that the framework 2 presents in any vertical plane a substantially U-shaped cross section as is clearly apparent in particular from FIG. 6.

The shell 3 of the dental aligner 1 is positioned on the framework 2, in particular accommodated or inserted between the labial 21, lingual 22 and occlusal 23 walls essentially. In other words, the framework 2 and the walls 21, 22, 23 thereof form a mould for supporting and forming the thermoformable shell 3 of the dental aligner 1.

The thermoformable shell 3 is constituted from a thermoplastic material that is of food grade and/or biocompatible quality. Preferably, the thermoformable shell 3 is made of ethylene-vinyl acetate, more commonly known by the acronym thereof EVA.

The thermoformable shell 3 is shaped so as to match the form of the rigid framework 2 and consequently thus has a U-shaped form analogous to that of the framework 2. The thermoformable shell 3 being shaped so as to match the form of the walls 21, 22, 23 of the framework 2 forms a covering chute for covering a dental arch of a patient which includes a labial wall 31 that rests against an internal surface of the labial wall 21 of the framework, a lingual wall 32 that rests against an internal surface of the lingual wall 22 of the framework 2 and a bottom wall 33 that rests against the internal surface of the occlusal wall 23 of the framework 2.

Thus, the labial walls 21, 31 and lingual walls 22, 32 are substantially parallel to each other and substantially perpendicular to the occlusal wall 23 and the bottom wall 33 of the thermoformable shell 3 in a manner such that the aligner 1 as a whole, comprising the framework 2 and the thermoformable shell 3 accommodated on the same has a substantially U-shaped cross section in any vertical plane, as is clearly apparent from FIGS. 1 to 7.

Preferably, in order to provide a greater degree of comfort of use of the dental aligner 1, the labial wall 31 and lingual wall 32 of the thermoformable shell 3 protrude out above the upper edge of the labial wall 21 and lingual wall 22 of the framework in a manner so as to form a support flange for supporting the thermoformable shell 3 on the framework 2 that is adapted so as to provide a foam contact surface with the buccal tissues of a patient during use of the dental aligner 1.

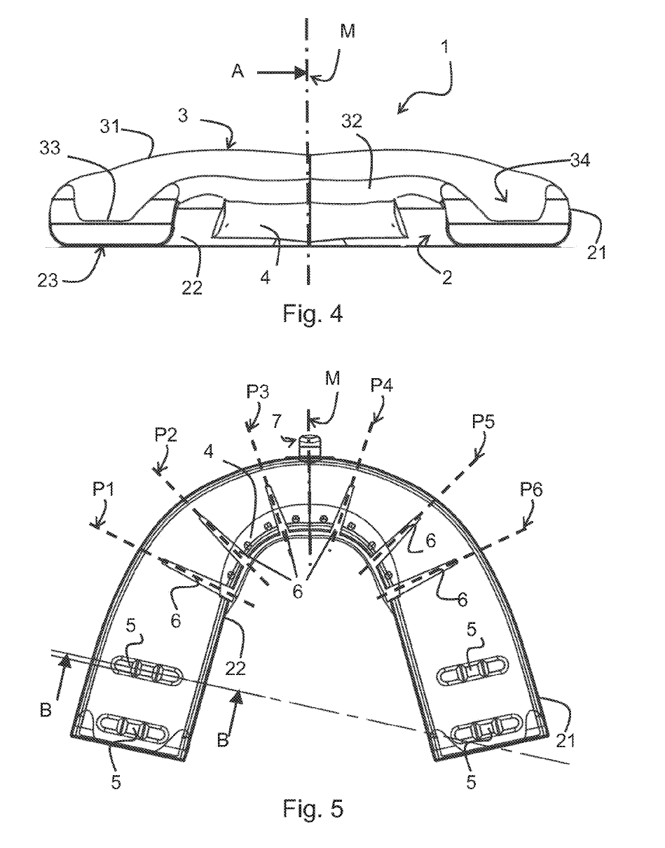

In accordance with the invention and as is clearly apparent from FIGS. 4 to 7 in particular, the thermoformable shell 3 of the dental aligner 1 is anchored on to the framework by way of anchoring means which are formed in the framework 2 and the shell 3. In particular, the anchoring means comprise at least one connecting tab 4 that is formed integrally together with the shell 3 made of thermoformable material and extending partially into a recess 6, in particular a slot, formed in at least the lingual wall 22 or only a portion thereof. Advantageously, the said recess 6 also constitutes a zone of flexion of the aligner the said framework 2 on both sides of at least one vertical plane M that is perpendicular to the occlusal wall 23 thereof. Preferably, the connecting tab 4 extends also in extension of the lingual wall 32, for example by over-moulding of the constituent material of the thermoformable shell 3 on the lingual wall of the framework 2.

Quite obviously, it may also be envisaged within the context of the invention to provide the connecting tab 4 not on the lingual wall 22 but on a portion or segment of the labial wall 21 of the framework 2 from the labial wall 31 of the shell 3 made of thermoformable material. It is finally also possible to provide a plurality of connecting tabs 4 covering at least partially at least a segment of the labial wall 21 and the lingual wall 22 of the framework in order to provide a plurality of anchoring points for anchoring the thermoformable shell 3 along the walls 21, 22 of the framework 2.

The implementation of a connecting tab 4 as described here above makes it possible to provide for a complete anchoring of the thermoformable shell 3 on the framework 2 as a result of the effect of crimping of the shell on the labial wall 21 and/or lingual wall 22 of the framework 2 by using the same material of the thermoformable shell 3 but in an extremely small amount in comparison with the total over-moulding techniques taught in the prior art. This enables considerably reducing the cost of materials in the production of the dental aligner of the invention, as well as the associated fabrication times.

In addition, the reduction of the material necessary for the over- moulding of the framework 2 provides for a significant reduction in the overall dimensional impact of the dental aligner 1 once fully constituted, which procures increased comfort of use for patients.

As represented in particular in FIGS. 5 and 7, the dental aligner 1 of the invention may also include by way of anchoring means, in combination or not, with the connecting tabs 4, the holes 5 formed through and through in the occlusal wall 23 of the framework 2, in particular preferably in the vicinity of the posterior ends of each of the lateral arms 2a, 2b of the framework 2. These holes 5 are conformed in order to allow, during the formation of the thermoformable shell 3 on the framework 2 and/or during form setting by the taking of an impression of a dental arch of a patient in the thermoformable shell 3 of the dental aligner 1 of the invention, an insertion of the thermoformable material of the shell 3 through the holes 5 from the internal surface of the occlusal wall 23 toward the external surface of this same occlusal wall 23, thereby forming anchoring pins for anchoring the shell 3 of the framework 2. The holes 5 are further conformed so as to prevent the withdrawing of the thermoformable material of the shell 3 from the external surface toward the internal surface of the occlusal wall 23. In this way, the thermoformable shell 3 is effectively anchored on the occlusal wall 23 of the framework 2 by means of anchoring pins made of thermoformable material in the holes 5, as it appears in particular in FIG. 7.

More precisely, and in an advantageous manner according to the invention the holes 5 are such that they have at least one dimension, taken in at least one vertical cross section of the occlusal wall 23 as represented in FIG. 7, which is smaller at the level of the internal surface of the occlusal wall 23 as compared to the corresponding dimension of the same hole at the level of the external surface of the occlusal wall 23. Preferably, the dimension of the holes 5 is thus variable on an increasing basis over the thickness of the occlusal wall 23 between the internal surface and the external surface of the said occlusal wall 23. According to this preferred principle of implementation, the holes 5 have a cross section, taken in the said vertical plane, with a dovetail-shaped form, that is to say at least partly triangular or trapezoidal. Alternatively a T-shaped cross section may also be envisaged.

The anchoring means of the thermoformable shell 3 of the dental aligner 1 of the invention provide for a highly effective connection of these two elements thereby preventing any loosening/dislodging of the thermoformable shell 3 from the framework 2 of the aligner 1 even in the event of extended use thereof and of strong mechanical stresses of chewing on the part of patients, while also making it possible as previously mentioned to achieve a significant reduction of the material of the thermoformable shell 3 and thus of the overall dimensional impact of the dental aligner 1, thereby promoting greater comfort of use for the patient with in particular fewer lesions of buccal tissues in contact with the dental aligner 1 as compared to the known aligners disclosed in the prior art.

In order to provide for the comfort and versatility of use for patients the framework 2 of the dental aligner 1 of the invention is preferably configured so as to be flexible on both sides of at least one vertical plane, in particular at least one mid sagittal plane M of the aligner 1 (FIGS. 1, 4, 5), which is perpendicular to the occlusal wall 23 of the framework 2. Even more preferably, the framework 2 is more broadly flexible on both sides of a plurality of vertical planes P1, P2, P3, P4, P5, P6 that are perpendicular to the occlusal wall 23 of the framework 2 as represented in FIG. 5.

In order to confer a flexible character to the framework with respect to one or more vertical plane as previously described, the said framework 2 comprises at least one favoured zone of flexion formed by localised reduction of the material of the framework 2, which for example extends in at least one said vertical plane on either side of which the framework 2 is flexible.

In one advantageous embodiment, the one or more favoured zone(s) of flexion of the framework 2 result at least from the recess 6 constituting an anchoring means, in particular a slot, formed in at least one labial or lingual wall and the occlusal wall of the framework. Preferably, the framework 2 comprises a plurality of recesses 6 formed exclusively in the lingual wall 22 and the occlusal wall 23 of the framework 2.

In one preferred embodiment as represented in FIG. 5 the framework 2 comprises a plurality of flexion zones formed by the recesses 6 or retreating of material located in the lingual wall 22 and occlusal wall 23 thereof. This embodiment provides for the best balance between flexibility and rigidity of the framework 2 in order to provide both for a degree of flexibility in adjustment to any type of conformation of a dental arch of a patient while also providing for a dental aligner 1 that is stable and equipped with a good elastic retention on the dental arch of the patient.

Another advantageous but accessory characteristic feature of the dental aligner 1 of the invention as represented in FIGS. 2-7 in particular lies in the integration, in the external face of the labial wall 21, of the framework 2, and in the median plane M of the aligner 1 of a pin or stud 7 projecting outward comprising a head 71 and a connecting foot 72 for connection to the labial wall 21.

This pin 7 provides an additional triple functionality to the dental aligner 1 of the invention as described here below with reference made to FIGS. 8 to 12.

As represented in FIG. 8 first of all, the pin 7 of the aligner 1 forms a blocking member for positioning the aligner 1 on an occlusal plate 9 in order to enable the dental impression - taking and the form setting of the aligner 1. For the sake of clarity, only the framework 2 of the dental aligner 1 is represented in FIG. 8 in order to show the particular positioning of the aligner on the occlusal plate 9.

The occlusal plate 9, advantageously provided with the aligner 1 of the invention in order to form a kit that provides the means to all patients to effectively set in form the aligner 1 in a non-clinic at-home setting, has a Y-shaped form of which the base forms a manual gripping arm 9a that is extended by two support arms 9b , 9c which are attached at one of their ends creating a V-shape that forms an acute angle at their base. These two V-shaped arms constitute support planes for supporting the dental aligner 1. On the support arms 9b , 9c are additionally also positioned, or preferably formed (for example by moulding the occlusal plate 9) wedging pads 9d projecting outwards over the support arms 9b , 9c of the said aligner 1. The wedging pads 9d , preferably being at a minimum 4 in number, are arranged in a manner so as to wedge the aligner 1 in position on the support arms 9b , 9c of the occlusal plate 9, in particular by supporting the lateral sections 2a, 2b of the framework 2 of the dental aligner 1 against at least one pad 9d each respectively on each support arm 9b , 9c , at the central section 2c of the framework 2 of the aligner 1 by insertion of the pin 7 of the framework 2 between two wedging pads 9d spaced away from each other by a distance substantially equally to the diameter of the foot 72 of the pin 7 in a plane perpendicular to the longitudinal axis of the said foot 72, which itself extends in the mid sagittal plane M of the aligner 1. The pin 7 in cooperation with the wedging pads 9d of the occlusal plate 9 enables a stable blocking in place of the aligner 1 of the occlusal plate 9 in order to execute the setting in form of the dental aligner according to a method described here below.

In order to do this, a patient needs to simply carry out the following steps. First of all, it is necessary to proceed with the heating of the dental aligner 1, for example by immersing the aligner 1 in its entirety in a water bath at a temperature comprised between 60 and 80° C. so as to soften the shell 3 made of thermoformable material, at least for a period of 1 min at a minimum, preferably for a period of 2 to 5 min.

Then, the dental aligner 1 is to be positioned on the occlusal plate 9 taking care to wedge the latter by insertion of the pin 7 between the wedging pads 9d in a manner so as to stabilise the dental aligner 1 for the impression taking.

Subsequently, in a third step the patient is to insert the occlusal plate 9 bearing the dental aligner 1 whose thermoformable shell 3 has been softened, in their mouth and thereafter proceed to the taking of the impression and the setting in form of the thermoformable shell 3 and the framework 2 of the dental aligner 1 by way of compressing the thermoformable shell 3 between a dental arch and the occlusal plate 9 by crimping of the jaw against the occlusal plate 9. Preferably, care should be taken to maintain the compressive force for a period of at least 20 seconds to one minute, thereafter the occlusal plate 9 and the dental aligner 1 thus set in form may then be removed from the mouth of the patient. As may be necessary, an additional step of cooling of the aligner may be envisaged in order to completely fix the form of the impression obtained, as well as to facilitate the subsequent removal of any burrs so as to provide for a perfect fit in the mouth of the patient during the use of the aligner.

During the setting in form of the dental aligner 1 the occlusal plate advantageously makes it possible to avoid leakage of the constituent thermoformable material of the shell 3 to the exterior of the framework 2 through the holes 5 and recesses or slots 6 of the said framework. This thereby prevents an increase in volume of the dental aligner 1 during the impression taking. In addition, the counter pressure produced by the occlusal plate 9 while the impression is being taken advantageously promotes the formation of anchoring pins for anchoring the shell 3 in the holes 5 provided for this purpose in the framework 2 by pressing down flatly on the material of the thermoformable shell 3 flowing through these holes 5 during the impression taking, which thus facilitates the distribution of the material in the holes, in particular into the sections of larger dimensions, which in turn prevents the retreating of material in these holes 5 during the cooling of the shell 3 made of thermoformable material.

Another function of the pin 7 of the framework 2 of the dental aligner 1 further also comprises of providing a centred and visible reference point on the aligner 1 so as to facilitate the centring of the latter during insertion thereof in the mouth of the patient for the impression taking. This can prove to be advantageous in the case of dental asymmetry relative to the mid sagittal anatomical plane of a patient.

The pin 7 finally provides a last accessory functionality to the aligner 1 of the present invention, which consists in enabling the attachment of a connecting strip for connecting of two dental aligners in the context of the embodiment of a mandibular orthosis as described here below with reference to FIGS. 9 to 12.

Mandibular orthoses are prescribed and used in a conventional manner in the context of a treatment for sleep apnea and comprise at least one mandibular dental aligner and a resilient elastic return system from this mandibular aligner to the front in order to avoid subsequent slippage of the jaw during sleep, which is a cause of respiratory pauses typical of sleep apnea. The elastic return system is in a conventional manner comprised of a band made of elastic material that is integrally fixed at its ends to the posterior anchor points on the mandibular aligner, the band being tensioned in the mouth of the patient against the maxillary arch of the patient.

In order to improve the comfort of patients using such mandibular orthoses it is preferable for the orthosis to comprise not one but two aligners, one mandibular aligner and one occlusal type dental aligner for the upper jaw, the two aligners then being connected to one another by an elastic band for exerting a propulsive force on the mandible, while also allowing for a certain freedom of movement.

A mandibular orthosis 100 having two dental aligners 101, 102 in accordance with the present invention that are connected to each other by an elastic band 103 is shown base on different views in FIGS. 9 through 12.

The upper dental aligner 101 of the mandibular aligner 100 is identical to the aligner shown in FIGS. 1 to 7. The lower dental aligner 102, or mandibular aligner differs from the upper dental aligner 101 only by the absence of the frontal pin 7. On the other hand the aligner 103 comprises two lateral pins 8 that are integrally attached to and protruding out over the framework of the aligner 103. These lateral pins 8 provide for the attachment and removal of the ends of the connecting band 103, each of which advantageously comprise holes provided for this purpose. The band 103 is in addition fixed by a hole also provided for this purpose substantially in its middle on the pin 7 of the upper aligner 101. This thus makes it possible to obtain an elastic band effect of the two aligners 101, 102 relative to each other in a manner such that the band 103 exerts, when the two aligners 101, 102 are positioned respectively, on the upper and lower jaw of a patient, a posterior-anterior traction force of the mandible in order to prevent any posterior sliding thereof during the various phases of sleep the patient undergoes, to thereby prevent sleep apnea.

During use, the external surfaces of the occlusal walls of the frames of each of the aligners 101, 102 are found to be in contact with one another along the occlusal plane, and may slide over each other.

The present invention thus proposes a dental aligner with reduced dimensional impact and enhanced comfort that is appropriate for treatment of bruxism and/or sleep apnea, whether it be applied in simple use with one single dental aligner conformed according to the form setting method of the invention for one of the jaws of a patient, or in the context of a mandibular orthosis having one or two dental aligners appropriately conformed according to the present invention and connected to each other by an elastic connecting band.

1. A dental aligner comprising a framework which is made of a rigid synthetic material and a shell made of thermoformable material, the framework having an anterior labial wall, and a posterior lingual wall, which walls are substantially parallel to each other and are connected by a bottom occlusal wall, the occlusal wall being substantially perpendicular to the labial and lingual walls in such a way that the framework has a U-shaped cross section in any vertical plane, wherein the shell is accommodated between the labial, lingual and occlusal walls of the framework, and wherein the shell is anchored thereon by way of anchoring means which are formed in the framework and the shell and having holes going therethrough from side to side between an internal surface of the occlusal wall on which rests the shell and an external surface of the occlusal wall.

2. The dental aligner according to claim 1, wherein the anchoring means comprise at least one connecting tab covering at least partially, at least one segment of the labial wall and/or the lingual wall, the said connecting tab being formed integrally together with the shell in a manner so as to crimp the at least one segment of the labial wall and/or the lingual wall between the shell and the connecting tab.

3. The dental aligner according to claim 1, wherein the holes have at least one dimension, taken in at least one vertical cross section of the occlusal wall, which is smaller at the level of the internal surface as compared to the corresponding dimension at the level of the external surface.

4. The dental aligner according to claim 3, wherein the holes have a dimension that is variable on an increasing basis over a thickness of the occlusal wall between the internal surface and the external surface of the occlusal wall.

5. The dental aligner according to claim 3, wherein the holes have a cross section, taken in the vertical cross section of the occlusal wall, that is at least partly triangular or trapezoidal.

6. A dental aligner, comprising a framework which is made of a rigid synthetic material and supports a shell made of thermoformable material, the framework having an anterior labial wall, and a posterior lingual wall, which walls are substantially parallel to each other and are connected by a bottom occlusal wall, the latter being substantially perpendicular to the labial and lingual walls in such a way that the framework has a U-shaped cross section in any vertical plane, wherein the shell is accommodated between the labial, lingual and occlusal walls of the framework, and wherein the shell is anchored to the framework by way of anchoring means that comprise at least one connecting tab extending partially into a recess, in particular a slot, formed in at least one of the labial wall and the lingual wall and in the occlusal wall of the framework, the connecting tab being formed integrally together with the shell so as to crimp the framework between the shell and the connecting tab, the recess constituting a zone of flexion of the aligner, the framework on both sides of at least one vertical plane being perpendicular to the occlusal wall.

7. The dental aligner according to claim 6, wherein the framework is flexible on both sides of a plurality of vertical planes that are perpendicular to the occlusal wall.

8. The dental aligner according to claim 7, wherein the framework comprises a plurality of recesses each extending in at least one of the plurality of vertical plane planes on either side of which the framework is flexible.

9. The dental aligner according to claim 8, wherein the plurality of recesses are formed exclusively in the lingual wall and the occlusal wall.

10. A form setting method for forming a dental aligner according to claim 1, comprising the following steps:
heating the dental aligner in a water bath at a temperature between 60° C. and 90° C. in order to soften the shell which is made of thermoformable material for a period of at least 1 minute;
positioning the dental aligner on an occlusal plate that is conformed substantially in a Y shape, the occlusal plate having one manual gripping arm and two support arms for supporting the dental aligner, which are attached at one of the ends thereof and at one end of the manual gripping arm, as well as wedging pads for wedging the dental aligner, which are fittingly adjusted on at least one face of the occlusal plate;
inserting the occlusal plate carrying the dental aligner in the mouth of a patient;
form setting the shell made of thermoformable material through application of pressure by one dental arch of the patient in the softened shell and counter pressure on the occlusal plate by the opposite dental arch in order to procure an effect of formwork and of penetration of the softened material of the shell into the holes and/or recess(s) of the anchoring means of the framework for forming the anchoring means; and
removing the occlusal plate and the dental aligner from the mouth of the patient.

11. The method according to claim 10, further comprising cooling the dental aligner after removal of the occlusal plate from the mouth of the patient.

12. The method according to claim 11, further comprising separating the shell and the framework after cooling.

13. The method according to claim 12, wherein separating the shell comprises the at least partial breaking of the anchoring means.

14. A mandibular orthosis comprising at least one dental aligner as defined in claim 1.
